(19) United States
(12) Patent Application Publication
(10) Pub. No.: US 2021/0046002 A1
(43) Pub. Date: Feb. 18, 2021

(54) AMELIORATION OF AUTOIMMUNE UVEITIS THROUGH BLOCKADE OF CSF1R

(71) Applicant: Massachusetts Eye and Ear Infirmary, Boston, MA (US)

(72) Inventors: Kip M. Connor, Newton, MA (US); Yoko Okunuki, Boston, MA (US)

(21) Appl. No.: 16/977,404

(22) PCT Filed: Mar. 4, 2019
(86) PCT No.: PCT/US2019/020539

§ 371 Date: Sep. 1, 2020

Related U.S. Application Data
(60) Provisional application No. 62/638,848, filed on Mar. 5, 2018.

(51) Int. Cl. A61K 9/00

(52) U.S. Cl. CPC A61K 9/0051; A61K 45/06; A61K 9/0014

(57) ABSTRACT

Methods and compositions for treating conditions including autoimmune uveitis using inhibitors of Colony stimulating factor 1 receptor (CSF1R).

### CLAIM OF PRIORITY

This application claims the benefit of U.S. Patent Application Ser. No. 62/638,848, filed on Mar. 5, 2018. The entire contents of the foregoing are hereby incorporated by reference.

### TECHNICAL FIELD

The present invention relates, at least in part, to methods and compositions for treating conditions including autoimmune uveitis using inhibitors of Colony stimulating factor 1 receptor (CSF1R).

### BACKGROUND

The uvea is the vascularized portion of the eye and is composed of the iris, ciliary body and, choroid. Autoimmune uveitis, represented by Behcet's disease, sarcoidosis, and Vogt-Koyanagi-Harada disease, is a sight-threatening ocular inflammatory disease [1, 2]. Although autoimmune uveitis covers a range of different clinical entities, autoimmunity against the retina and the uveal tissues is thought to be the main pathogenesis [3].

### SUMMARY

Provided herein are methods and compositions for treating autoimmune uveitis in a subject, using a therapeutically effective amount of a CSF1R inhibitor.

In some embodiments, the inhibitor of CSF1R is selected from the group consisting of PLX647; Ki20227; Pexidartinib (PLX3397, PLX108-01); PLX7486; OSI-930; Linifanib (ABT-869); ARRY-382; JNJ-40346527; GW2580; GTP 14564; AAL-993; and BLZ945, all of which are commercially available. Therapeutic antibodies include Emactuzumab (RG7155); AMG820; IMC-CS4 (LY3022855); and cabiralizumab (see, e.g., US2008/073611; US2011/030148); imatinib also has weak activity against CSF1R. See, e.g., Ries et al., Cancer Cell 25 (6):846-859; Cannarile et al., J Immunother Cancer. 2017; 5: 53.

In some embodiments, inhibitor is administered locally to the eye, e.g., administered topically or periocularly.

In some embodiments, the inhibitor is administered systemically, e.g., orally or parenterally.

Also provided herein are CSF1R inhibitors for use in treating autoimmune uveitis in a subject, e.g., selected from the group consisting of PLX647; Ki20227; Pexidartinib; PLX7486; OSI-930; Linifanib; ARRY-382; JNJ-40346527; GW2580; GTP 14564; AAL-993; BLZ945; Emactuzumab; AMG820; IMC-CS4; and cabiralizumab. In some embodiments, the CSF1R inhibitor is formulated administration to the eye, e.g., for topical or periocular administration, or for systemic administration.

Supplementary active compounds can also be administered and/or incorporated into the compositions, e.g., corticosteroids; antimetabolites (e.g., methotrexate, azathioprine, or mycophenolate mofetil); alkylating/cytotoxic agents (e.g., cyclophosphamide or chlorambucil); T cell and calcineurin inhibitors (e.g., cyclosporine or FK506/Tacrolimus); IVIG; and immunosuppressant biologicals including anti-TNF antibodies (e.g., Infliximab, Adalimumab, or Etanercept) IL-2R antagonists (e.g., Daclizumab) (see Papotto et al., Autoimmun Rev. 2014 September; 13 (9): 909-916).

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-D. Microglia depletion by a small molecule CSFR1 inhibitor suppresses EAU. C57BL/6 mice were fed with a small molecule CSFR1 inhibitor or control diet starting at day −7 and induced EAU on day 0. (A) Time course clinical score and (B) the representative retinal images on day 21 (n=7). (C) Histopathological score and (D) representative images on day 21 (n=5). Scale bars, 100 μm. (A, C) Mann-Whitney's test. Data are expressed as mean±s.e.m. The significance levels are marked *P<0.05; **P<0.01; ***P<0.001.

FIGS. 2A-E. Microglia depletion by a small molecule CSFR1 inhibitor in EAU does not alter systemic immune response to the immunized peptide.

EAU was induced in C57BL/6 mice fed with a small molecule CSFR1 inhibitor or control diet. (A) Delayed hypersensitivity evaluated on day 21 (n=6-7). (B-E) Cell proliferation evaluated by MTT assay in lymph nodes and spleens on day 14 and 21 (n=5). One-way ANOVA followed by Tukey's multiple comparison test. Data are expressed as mean±s.e.m. The significance levels are marked *P<0.05; **P<0.01; ***P<0.001; ****P<0.0001. LN, lymph node; SP, spleen; ConA, concanavalin A.

FIGS. 3A-F. A small molecule CSFR1 inhibitor does not suppress cytokine productions from LNs and SPs in EAU but suppresses CD11c⁺CD11b⁺ myeloid lineage cells.

LN cells and SP cells from EAU mice fed with a small molecule CSFR1 inhibitor or control diet and naïve mice were analyzed by flowcytometry on day 14. (A, D) CD11b and CD11c expression on CD45⁺ cells, (B, E) IFN-γ and IL-17 expression on CD3⁺CD4⁺ cells, and (C, F) CD25⁺ Foxp3⁺ cells on CD3⁺CD4⁺ cells in LNs (A-C) and in SPs (D-F). All n=5. Data were analyzed by one-way ANOVA followed by Tukey's multiple comparison test. Data are expressed as mean±s.e.m. The significance levels are marked *P<0.05; **P<0.01; ***P<0.001; ****P<0.0001. LN, lymph node; SP, spleen.

FIGS. 4A-E. A small molecule CSFR1 inhibitor suppresses EAU in recipient mice but does not significantly suppress uveitogenicity of donor lymphocytes in adoptive transfer EAU models. Adoptive transfer EAU was induced in recipient mice by transferring the activated lymphocytes from donor mice which had been induced EAU by active immunization. (A-C) a small molecule CSFR1 inhibitor was given to recipient mice 7 days prior to cell transfer from donor mice. (A) A schematic time course of the experiment in which a small molecule CSFR1 inhibitor was given in recipient mice. (B) Time course clinical score and representative fundus images of day 13 (n=5). (C) Histopathological score on day 13 and representative images of the recipient PLX experiment (n=5). (D, E) a small molecule CSFR1 inhibitor was given to donor mice 7 days prior to induction of EAU and then the cells were transferred to the recipient mice with regular diet. (D) A schematic time course of the experiment in which a small molecule CSFR1 inhibitor was given in donor mice. (E) Time course of clinical score and representative fundus images of day 14 in the donor PLX experiment (n=5). Mann-Whitney's test. Data are expressed as mean±s.e.m. The significance levels are marked *P<0.05; **P<0.01. Scale bars, 200 μm.

FIGS. 5A-D. Microglia depletion in CX3CR1^{creER}-iDTR transgenic (TG) mice suppresses EAU. (A) Evaluation of microglia depletion in TG mice. One-way ANOVA followed by Dunnet's multiple comparison test. (B) A schematic time course of adoptive transfer experiment in TG mice. (C) Clinical score on day 10 and representative fundus images. Mann-Whitney's test. N=5-6. (D) Representative histopathology of the eyes on day 10. Scale bars, 200 μm. Data are expressed as mean±s.e.m. The significance levels are marked *P<0.05; ***P<0.001. Tam, tamoxifen; DTX, diphtheria toxin; n.s., not significant.

FIGS. 6A-D. Microglia depletion after EAU development does not decrease inflammation. C57BL/6 mice were fed with a small molecule CSFR1 inhibitor or control diet starting at various time points and evaluated for clinical score. (A) A schema of time course of diet, EAU induction, and evaluation. (B-D) Time course clinical score. PLX or control diet was started on day 0 (B), day 7 (C), and day 14 (D) of EAU induction. (B) n=7-8, (C) n=7, (D) n=8-9. Mann-Whitney's test. Data are expressed as mean±s.e.m. The significance levels are marked *P<0.05; **P<0.01; ***P<0.001.

FIGS. 7A-D. Adhesion molecules in the retinal vessels are upregulated but the number of adhesive leukocytes are decreased in EAU of microglia depleted retina. (A, B) Mice fed with the control or a small molecule CSFR1 inhibitor diet were induced EAU. On day 10, retinal adherent leukocytes were imaged by perfusion labeling with FITC concanavalin A lectin. (A) Representative images of flatmounted retinas from each group are presented. Images are around the optic disc (top) and the mid-periphery (bottom). Adherent leukocytes are indicated by arrows. Scale bars, 50 μm. (B) Mean number of adherent retinal leukocytes in major vessels per eye. n=6-8. (C, D) Retinal protein obtained from Naïve mouse on the control diet and the EAU mice on the control or a small molecule CSFR1 inhibitor diet were subjected to western blot on day 10 and 14. Results were semiquantified by densitometry and normalized by b-actin levels. (C) Data were expressed as fold change against Naive. n=5-6. Data were analyzed by one-way ANOVA followed by Tukey's multiple comparison test and expressed as mean±s.e.m. The significance levels are marked *P<0.05; **P<0.01; ***P<0.001; ****P<0.0001.

FIGS. 8A-B. A small molecule CSFR1 inhibitor does not change lymph node and spleen weight in EAU. C57BL/6 mice were induced EAU by active immunization and weight of draining lymph nodes and spleens were measured on day 21. (A) Lymph node weight (n=8-9). (B) Spleen weight (n=5). Data were analyzed by one-way ANOVA followed by Tukey's multiple comparison test. Data are expressed as mean±s.e.m. The significance levels are marked ****P<0.0001.

FIGS. 9A-C. Tamoxifen suppresses EAU when it is given systemically. (A) A schematic figure of the timing of tamoxifen treatment and EAU induction in CX3CR1^{creER}-iDTR mice. (B) Clinical EAU score and representative fundus images of day 21 when tamoxifen or vehicle was given intraperitoneally. n=9 (C) Clinical EAU score and representative fundus images of day 21 when tamoxifen or vehicle was given via eye drops. n=6-8. Mann-Whitney's U test. Data are expressed as mean±s.e.m. The significance levels are marked ****P<0.0001.

FIGS. 10A-B. Microglia change their morphology in EAU. EAU was induced in C57BL/6 mice and then whole mount retinas were stained with anti-P2ry12 Ab at 0, 7, 10 and 14 days after EAU induction. (A) Area of P2ry12+ microglia (B) The number of P2ry12+ microglia in the midperipheral retina. n=4. Two images of different areas of a retina were used to calculated the area and microglia number of the retina. Data were analyzed by one-way ANOVA followed by Tukey's multiple comparison test. Data are expressed as mean±s.e.m. The significance levels are marked ****P<0.0001.

### DETAILED DESCRIPTION

Experimental autoimmune uveitis (EAU) is an animal model of human autoimmune uveitis and has been widely used for dissecting mechanisms and developing treatment strategies [4]. EAU is induced by immunization with retinal antigens such as interphotoreceptor retinoid-binding protein (IRBP), which is a major component of the outer segment photoreceptor cells that are presumed to be the primary autoimmune target in EAU [5, 6]. Immunization with IRBP in adjuvant context leads to priming of autoreactive T cells in peripheral lymphoid organs and polarization into Th1 and Th17 cells. Once activated Th cells home to the eye, they induce blood-retinal barrier (BRB) breakdown. After the initial Th cell entry in the retina, either resident retinal cells, such as microglia or perivascular macrophages, or infiltrating hematopoietic cells play a role of antigen presenting cells (APCs) and stimulate Th cells in the retina [7-9]. Subsequently massive recruitment of diverse inflammatory leukocytes from the circulation follows. Upregulation of adhesion molecules, such as ICAM-1 and VCAM-1, in the retinal vessels concurrent with the expression of their ligands on the leukocytes is thought to be the key for leukocyte entry into the retina [10, 11].

Microglia are resident immune cells of the central nervous system/retina and function in the homeostatic maintenance of the neuro-retinal microenvironment [12], while they are also an important component of neovascular unit (NVU). Microglia become activated during various retinal disease processes [13-21] including autoimmune [22] and non-autoimmune uveitis [22, 23]. It has been established that activated microglia enhance multiple functions such as phagocytosis, antigen presentation and production of inflammatory factors, which can be either beneficial or harmful to the affected tissue [24, 25].

Since microglia express MHC-class II molecules during the course of EAU, the role of microglia as APCs has long been investigated [7, 8, 26]. However, the exact role of microglia in autoimmune uveitis is still unknown. Some of the difficulties in the past studies were lack of microglia specific markers and depletion method, since microglia share common markers with monocytes/macrophages [27]. In this study, in order to elucidate the function of microglia in autoimmune uveitis, we applied a newly available microglia specific marker P2ry12 [28] and also a diet containing a small molecule CSFR1 inhibitor, a colony stimulating factor-1 receptor (CSF1R) antagonist that specifically depletes microglia [29, 30]. Here we report that microglia are essential for induction of EAU without expressing MHC class II and suggest that microglia play a key role of introducing inflammatory cells in the retina in the very beginning stage of inflammation.

The study described herein demonstrated that EAU was dramatically suppressed by microglia depletion by CSF1R inhibition. Without wishing to be bound by theory, it appears that the effect of CSF1R inhibition on EAU suppression was locally elicited by microglia depletion, not by systemic effect of CSF1R blockage. The present images of microglia from EAU animals suggest that microglia contribute in induction of EAU by supporting cell adhesion and possibly by promoting cell entry into the retina.

### Methods of Treatment

The methods described herein include methods for the treatment of disorders associated with microglial activation, e.g., autoimmune uveitis, e.g., in a mammal, e.g., in a human or non-human mammal (e.g., a veterinary or zoological subject). In some embodiments, the disorder is Behcet's disease, sarcoidosis, or Vogt-Koyanagi-Harada disease, each of which can be diagnosed using methods known in the art; see, e.g., International Team for the Revision of the International Criteria for Behçet's Disease (ITR-ICBD) et al., J Eur Acad Dermatol Venereol. 2014; 28: 338-347; Heinle and Chang, Autoimmun Rev. 2014 April-May; 13 (4-5):383-7; and Read et al., Am J Ophthalmol. 2001 May; 131 (5):647-52, respectively. The methods can also be used to deplete microglia to ameliorate or treat other diseases, e.g., encephalitis. Generally, the methods include administering a therapeutically effective amount of a CSF1R inhibitor as described herein, to a subject who is in need of, or who has been determined to be in need of, such treatment. In some embodiments, the methods can include administering a standard treatment for autoimmune uveitis, e.g., administering a therapeutically effective amount of one or more immunosuppressive agents, e.g., corticosteroids; antimetabolites (e.g., methotrexate, azathioprine, or mycophenolate mofetil); alkylating/cytotoxic agents (e.g., cyclophosphamide or chlorambucil); T cell and calcineurin inhibitors (e.g., cyclosporine or FK506/Tacrolimus); IVIG; and immunosuppressant biologicals including anti-TNF antibodies (e.g., Infliximab, Adalimumab, or Etanercept) IL-2R antagonists (e.g., Daclizumab) (see Papotto et al., Autoimmun Rev. 2014 September; 13 (9): 909-916).

As used in this context, to “treat” means to ameliorate at least one symptom of the disorder associated with microglial activation, e.g., autoimmune uveitis. Often, microglial activation in autoimmune uveitis results in blurred vision, photophobia, eye pain, floaters, headache and conjunctival injection; thus, a treatment can result in a reduction in blurred vision, photophobia, eye pain, floaters (floating spots), headache and conjunctival injection and a return or approach to normal vision. See, e.g., Amador-Patarroyo et al., Chapter 37: Autoimmune uveitis, in Autoimmunity: From Bench to Bedside; Anaya J M, Shoenfeld Y, Rojas-Villarraga A, et al., editors. Bogota (Colombia): El Rosario University Press; 2013 Jul. 18. Administration of a therapeutically effective amount of a compound described herein for the treatment of a condition associated with microglial activation in autoimmune uveitis will result in decreased levels of inflammation and an improvement, or reduction in progression or rate of progression, of symptoms associated with microglial activation in autoimmune uveitis.

### CSF1R Inhibitors

A number of CSF1R inhibitors are known in the art, and include small molecules, inhibitory nucleic acids, and inhibitory antibodies.

Small molecule inhibitors of CSF1R include PLX647; Ki20227; Pexidartinib (PLX3397, PLX108-01); PLX5622; PLX7486; PLX73086; OSI-930; Linifanib (ABT-869); ARRY-382; JNJ-40346527; GW2580; GTP 14564; AAL-993; and BLZ945, all of which are commercially available. Therapeutic antibodies include Emactuzumab (RG7155); AMG820; IMC-CS4 (LY3022855); and cabiralizumab (see, e.g., US2008/073611; US2011/030148); imatinib also has weak activity against CSF1R. See, e.g., Ries et al., Cancer Cell 25 (6):846-859; Cannarile et al., J Immunother Cancer. 2017; 5: 53. MSC110, or other CSF1 inhibitors, can also be used.

Inhibitory nucleic acids targeting CSF1R can also be used. Inhibitory nucleic acids useful in the present methods and compositions include antisense oligonucleotides, ribozymes, external guide sequence (EGS) oligonucleotides, siRNA compounds, single- or double-stranded RNA interference (RNAi) compounds such as siRNA compounds, modified bases/locked nucleic acids (LNAs), peptide nucleic acids (PNAs), and other oligomeric compounds or oligonucleotide mimetics which hybridize to at least a portion of the target nucleic acid and modulate its function. In some embodiments, the inhibitory nucleic acids include antisense RNA, antisense DNA, chimeric antisense oligonucleotides, antisense oligonucleotides comprising modified linkages, interference RNA (RNAi), short interfering RNA (siRNA); a micro, interfering RNA (miRNA); a small, temporal RNA (stRNA); or a short, hairpin RNA (shRNA); small RNA-induced gene activation (RNAa); small activating RNAs (saRNAs), or combinations thereof. See, e.g., WO 2010040112.

In some embodiments, the inhibitory nucleic acids are 10 to 50, 10 to 20, 10 to 25, 13 to 50, or 13 to 30 nucleotides in length. One having ordinary skill in the art will appreciate that this embodies inhibitory nucleic acids having complementary portions of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length, or any range therewithin. In some embodiments, the inhibitory nucleic acids are 15 nucleotides in length. In some embodiments, the inhibitory nucleic acids are 12 or 13 to 20, 25, or 30 nucleotides in length. One having ordinary skill in the art will appreciate that this embodies inhibitory nucleic acids having complementary portions of 12, 13, 14, 15, 16, 17, 18, 19, 20, 21, 22, 23, 24, 25, 26, 27, 28, 29 or 30 nucleotides in length, or any range therewithin (complementary portions refers to those portions of the inhibitory nucleic acids that are complementary to the target sequence).

The inhibitory nucleic acids useful in the present methods are sufficiently complementary to the target RNA, i.e., hybridize sufficiently well and with sufficient specificity, to give the desired effect. “Complementary” refers to the capacity for pairing, through hydrogen bonding, between two sequences comprising naturally or non-naturally occurring bases or analogs thereof. For example, if a base at one position of an inhibitory nucleic acid is capable of hydrogen bonding with a base at the corresponding position of a RNA, then the bases are considered to be complementary to each other at that position. 100% complementarity is not required.

Routine methods can be used to design an inhibitory nucleic acid that binds to the target sequence with sufficient specificity. In some embodiments, the methods include using bioinformatics methods known in the art to identify regions of secondary structure, e.g., one, two, or more stem-loop structures, or pseudoknots, and selecting those regions to target with an inhibitory nucleic acid. For example, “gene walk” methods can be used to optimize the inhibitory activity of the nucleic acid; for example, a series of oligonucleotides of 10-30 nucleotides spanning the length of a target RNA can be prepared, followed by testing for activity. Optionally, gaps, e.g., of 5-10 nucleotides or more, can be left between the target sequences to reduce the number of oligonucleotides synthesized and tested. GC content is preferably between about 30 60%. Contiguous runs of three or more Gs or Cs should be avoided where possible (for example, it may not be possible with very short (e.g., about 9-10 nt) oligonucleotides).

In some embodiments, the inhibitory nucleic acid molecules can be designed to target a specific region of the CSF1R sequence; an exemplary sequence for human CSF1R can be found in GenBank at Acc. No. NP_001275634.1. For example, a specific functional region can be targeted, e.g., promoter or enhancer region. Alternatively or in addition, highly conserved regions can be targeted, e.g., regions identified by aligning sequences from disparate species such as primate (e.g., human) and rodent (e.g., mouse) and looking for regions with high degrees of identity. Percent identity can be determined routinely using basic local alignment search tools (BLAST programs) (Altschul et al., J. Mol. Biol., 1990, 215, 403-410; Zhang and Madden, Genome Res., 1997, 7, 649-656), e.g., using the default parameters.

Once one or more target regions, segments or sites have been identified, e.g., within a target sequence known in the art or provided herein, inhibitory nucleic acid compounds are chosen that are sufficiently complementary to the target, i.e., that hybridize sufficiently well and with sufficient specificity (i.e., do not substantially bind to other non-target RNAs), to give the desired effect.

In the context of this invention, hybridization means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds. Complementary, as used herein, refers to the capacity for precise pairing between two nucleotides. For example, if a nucleotide at a certain position of an oligonucleotide is capable of hydrogen bonding with a nucleotide at the same position of a RNA molecule, then the inhibitory nucleic acid and the RNA are considered to be complementary to each other at that position. The inhibitory nucleic acids and the RNA are complementary to each other when a sufficient number of corresponding positions in each molecule are occupied by nucleotides which can hydrogen bond with each other. Thus, “specifically hybridisable” and “complementary” are terms which are used to indicate a sufficient degree of complementarity or precise pairing such that stable and specific binding occurs between the inhibitory nucleic acid and the RNA target. For example, if a base at one position of an inhibitory nucleic acid is capable of hydrogen bonding with a base at the corresponding position of a RNA, then the bases are considered to be complementary to each other at that position. 100% complementarity is not required.

It is understood in the art that a complementary nucleic acid sequence need not be 100% complementary to that of its target nucleic acid to be specifically hybridisable. A complementary nucleic acid sequence for purposes of the present methods is specifically hybridisable when binding of the sequence to the target RNA molecule interferes with the normal function of the target RNA to cause a loss of activity, and there is a sufficient degree of complementarity to avoid non-specific binding of the sequence to non-target RNA sequences under conditions in which specific binding is desired, e.g., under physiological conditions in the case of in vivo assays or therapeutic treatment, and in the case of in vitro assays, under conditions in which the assays are performed under suitable conditions of stringency. For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

In general, the inhibitory nucleic acids useful in the methods described herein have at least 80% sequence complementarity to a target region within the target nucleic acid, e.g., 90%, 95%, or 100% sequence complementarity to the target region within an RNA. For example, an antisense compound in which 18 of 20 nucleobases of the antisense oligonucleotide are complementary, and would therefore specifically hybridize, to a target region would represent 90 percent complementarity. Percent complementarity of an inhibitory nucleic acid with a region of a target nucleic acid can be determined routinely using basic local alignment search tools (BLAST programs) (Altschul et al., J. Mol. Biol., 1990, 215, 403-410; Zhang and Madden, Genome Res., 1997, 7, 649-656). Inhibitory nucleic acids that hybridize to an RNA can be identified through routine experimentation. In general the inhibitory nucleic acids must retain specificity for their target, i.e., must not directly bind to, or directly significantly affect expression levels of, transcripts other than the intended target.

For further disclosure regarding inhibitory nucleic acids, please see US2010/0317718 (antisense oligos); US2010/0249052 (double-stranded ribonucleic acid (dsRNA)); US2009/0181914 and US2010/0234451 (LNAs); US2007/0191294 (siRNA analogues); US2008/0249039 (modified siRNA); and WO2010/129746 and WO2010/040112 (inhibitory nucleic acids).

### Pharmaceutical Compositions and Methods of Administration

The methods described herein include the use of pharmaceutical compositions comprising CSF1R inhibitors as an active ingredient.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions, e.g., corticosteroids; antimetabolites (e.g., methotrexate, azathioprine, or mycophenolate mofetil); alkylating/cytotoxic agents (e.g., cyclophosphamide or chlorambucil); T cell and calcineurin inhibitors (e.g., cyclosporine or FK506/Tacrolimus); IVIG; and immunosuppressant biologicals including anti-TNF antibodies (e.g., Infliximab, Adalimumab, or Etanercept) IL-2R antagonists (e.g., Daclizumab) (see Papotto et al., Autoimmun Rev. 2014 September; 13 (9): 909-916).

Pharmaceutical compositions are typically formulated to be compatible with its intended route of administration. Examples of routes of administration include parenteral, e.g., intravenous, intradermal, subcutaneous, oral (e.g., inhalation), transdermal (topical), transmucosal, and rectal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, N.Y.). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798.

Therapeutic compounds that are or include nucleic acids can be administered by any method suitable for administration of nucleic acid agents, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al., Clin. Immunol. Immunopathol., 88 (2), 205-10 (1998). Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. Nanoparticles (1 to 1,000 nm) and microparticles (1 to 1,000 μm), e.g., nanospheres and microspheres and nanocapsules and microcapsules, can also be used. These can be prepared according to methods known to those skilled in the art, for example, as described in U.S. Pat. No. 4,522,811; Bourges et al., Ocular drug delivery targeting the retina and retinal pigment epithelium using polylactide nanoparticles. Invest Opth Vis Sci 44:3562-9 (2003); Bourges et al., Intraocular implants for extended drug delivery: therapeutic applications. Adv Drug Deliv Rev 58:1182-1202 (2006); Ghate et al., Ocular drug delivery. Expert Opin Drug Deliv 3:275-87 (2006); and Short, Safety Evaluation of Ocular Drug Delivery Formulations: Techniques and Practical Considerations. Toxicol Pathol 36 (1):49-62 (2008).

The pharmaceutical compositions can be included in a container, pack, or dispenser together with instructions for administration.

### EXAMPLES

The invention is further described in the following examples, which do not limit the scope of the invention described in the claims.

### Materials and Methods

The following materials and methods were used in the examples below.

### Mice, Reagents and Monoclonal Antibodies

All animal experiments followed the guidelines of the ARVO Statement for the Use of Animals in Ophthalmic and Vision Research and were approved by the Animal Care Committee of the Massachusetts Eye and Ear Infirmary. C57BL/6J mice (stock no. 00664) and CX3CR1^{GFP/GFP} mice on a C57BL/6 background (stock no. 005582), Cx3cr1^{CreER} mice (stock no. 021160), and B6-iDTR mice (stock no. 007900) were purchased from Jackson Laboratories (Bar Harbor, Me., USA). Heterozygous CX3CR1^{+/GFP} mice were created by crossing CX3CR1^{GFP/GFP} mice with wild-type C57BL/6J mice. Standard laboratory chow was fed to mice except during the microglia depletion experiments, in which a small molecule CSFR1 inhibitor or the control diet was given. Mice were allowed free access to water in an air-conditioned room with a 12-hour light/12-hour dark cycle. All mice used for experiments were 7-9 weeks old. For anesthesia, intraperitoneal (i.p.) injection of 250 mg/kg of 2,2,2-tribromoethanol (Sigma-Aldrich Corp., St. Louis) was used for survival procedures and 400 mg/kg was used for retinal perfusion and enucleation.

High pressure liquid chromatography-purified human interphotoreceptor retinoid binding protein peptide 1-20 (IRBP-p) was purchased from Biomatik (Wilmington, Del.). Complete Freund's Adjuvant (CFA) and Mycobacterium tuberculosis H37Ra were purchased from Difco (Detroit, Mich.). Purified Bordetella pertussis toxin (PTX), Phorbol 12-myristate 13-acetate (PMA), ionomycin, Histopaque 1083, penicillin and streptomycin, were purchased from Sigma-Aldrich (St. Louis, Mo.).

### Induction of EAU

For active induction of EAU, 200 μg of IRBP-p 1-20 was emulsified in CFA (1:1 w/v) containing additional 5 mg/ml M. tuberculosis H37Ra. On day 0, 200 μl of the emulsion was injected subcutaneously in the neck (100 μl), one footpad (50 μl) and the other side of inguinal legion (50 μl). Concurrent with immunization, 1 μg of PTX was injected intraperitoneally (i.p.).

Adoptive transfer EAU was induced as previously described [62] with brief modification. Briefly, donor mice were immunized as described above and the spleens and draining lymph nodes (LNs) were collected on day 14 post-immunization. Lymphocytes from spleens and draining LNs were culture in the presence of 10 μg/ml IRBP-p and 10 ng/ml IL-23 (R&D systems, Minneapolis, Minn.) for 72 hours in RPMI 1640 supplemented with 10% FBS (Gibco), 2 mM glutamine (Gibco), and 100 U/ml penicillin and 100 μg/ml streptomycin. The non-adherent cells in the entire suspension were transferred to new dishes on day one and two of culture. After 3 days, the activated lymphocytes were purified by gradient centrifugation on Histopaque 1083 and counted. The cells were injected i.p. in 0.2 ml of PBS into donor mice (5×10⁷ cells/mouse).

### Assessment of EAU

Fundus were observed using Micron IV (Phoenix, Pleasanton, Calif.) and clinical score was graded on a scale between 0-4 in half-point increments as described previously [63]. For histological assessment, enucleated eyes were fixed in a buffer of 70% methanol and 30% acetic acid. The fixed tissues were embedded in paraffin and processed. Sections of 5 μm were cut and stained with H&E. The severity of EAU in each eye was scored on a scale between 0-4 in half-point increments, according to a semiquantitative system described previously [64].

### Delayed Hypersensitivity Measurement

Ag-specific delayed hypersensitivity (DH) was measure as previously described [65]. On day 19 after immunization, mice were injected intradermally with 10 μg/10 μl of IRBP-p suspended in PBS into the pinna of one ear. Ear swelling was measured after 24 and 48 h using a micrometer (Mitutoyo, Tokyo, Japan). DH was measured as the difference in ear thickness before and after challenge. Results were expressed as: specific ear swelling=(24-h measurement−0-h measurement) for test ear−(24-h measurement−0-h measurement) for control ear.

### Immunohistochemistry of Whole Mount Retinas

The mice were perfused with 20 mL of PBS after anesthesia. The eyes were enucleated and fixed in 4% paraformaldehyde in 2×PBS for 15 minutes, then transferred to 2×PBS on ice for 10 minutes. After dissecting the eyes, retinal wholemounts were prepared. The retinas were then transferred to ice cold methanol and kept at −80° C. until use.

For immunohistochemistry, the retinas were first blocked in a blocking buffer (0.3% Triton, 0.2% BSA, and 5% goat serum in PBS) for 1 hour at room temperature and incubated with 1^{st} antibodies and Alexa Flour® 647 conjugated Isolectin GS-B4 (1:100, Thermo Fisher Scientific) over night at 4° C. After washing, the retinas were incubated with 2^{nd} antibodies for 4 hours at 4° C. The retinas were mounted after washing. Rabbit anti-P2ry12 Ab (1:500; a gift from H. Weiner, Brigham and Women's hospital), rat anti-CD11b Ab (1:100, clone M1/70, abcam, Cambridge, Mass., USA), rat anti-F4/80 Ab (1:2000, clone CI:A3-1, Bio-Rad, Raleigh, N.C., USA), rat anti-ICAM-1 (1:200, clone YN1/1.7.4, Biolegend), rat anti-VCAM-1 (1:200, clone 429, Biolegend) were used for 1^{st} antibodies and Alexa Flour® 594-conjugated goat anti-rabbit Ab, and Alexa Flour® 488-conjugated goat anti-rat Ab (1:500, Thermo Fisher Scientific, Waltham, Mass., USA) were used for 2^{nd} antibodies.

### Lectin Labeling of Adherent Retinal Leukocytes

The retinal vasculature and adherent leukocytes were imaged by perfusion labeling with fluorescein-isothiocyanate (FITC)-conjugated concanavalin A lectin (conA; Vector Laboratories, Burlingame, Calif.), as described previously with modifications [66, 67]. Briefly, after deep anesthesia, the chest cavity was opened and a 27-gauge cannula was introduced into the left ventricle. The mice were perfused through the left ventricle first using 5 ml of PBS, followed by fixation with 1% paraformaldehyde (5 ml), FITC-conjugated conA (20 μg/ml in PBS, 5 mL), and 5 ml of PBS. The eyes were then fixed in 4% PFA for 15 mins and the retinas were flatmounted. The total number of conA stained adherent leukocytes in the major retinal vessels (venules, arterioles, and collecting vessels) were counted under the direct observation with an epifluorescent microscopy (Axio Observer Z1; Carl Zeiss, Bayern, Germany).

### Image Processing and Analysis

The images of whole mount retinas were taken by a confocal microscopy (SP5 or SP8; Leica, Buffalo Grove, Ill., USA) or an epifluorescent microscopy (Axio Observer Z1; Carl Zeiss, Bayern, Germany). For microglial cell number counting, microglial cell bodies were manually counted based on the z-stack images. For microglial density evaluation, maximum intensity z-stack images were created and the images were processed with the smooth, the make binary, and the watershed tools. The area of particles was then calculated using the analyze particles tool, setting the size range to 5-1000. Amira 5 software (FEI, Hillsboro, Oreg., USA) was used to make 3D-reconstruction images.

### Western Blot

Whole retinas were homogenized with a hand homogenizer and briefly sonicated in M-PER® Mammalian Protein Extract Reagent containing Halt Phosphatase Inhibitor Cocktail (both Thermo Scientific), and then ultracentifuged for 15 min at 17,000 g to collect soluble proteins as supernatants. 20 μg of the samples were electrophoresed through 4-15% polyacrylamide gels (Bio-Rad, Hercules, Calif., USA), and transferred onto PVDF membranes. After blocking with 5% nonfat dried milk, the membranes were incubated overnight with a primary antibody at 4° C. The membranes were then incubated for 1 hour at room temperature with a HRP-labeled secondary antibody. The following antibodies were used: rabbit anti-ICAM1 (179707, abcam), rabbit anti-VCAM1 (134047, abcam), and rabbit anti-β-actin (4970; Cell Signaling Technology). Immunoreactive bands were visualized by ECL. The images were taken by ChemiDoc MP (Bio-Rad) and analyzed using Image Lab 4.6 (Bio-Rad).

### Flow Cytometric Analysis of Lymph Nodes and Spleens

Cervical, axillary, and inguinal lymph nodes (LNs) were harvested from naive mice and EAU mice fed with a small molecule CSFR1 inhibitor or control diet. Single cell suspensions (1×10⁶ cells/sample) were blocked with anti-mouse CD16/32 mAb (eBioscience, San Diego, Calif.) and stained with cell surface antibodies. Dead cells were stained with LIVE/DEAD™ fixable dead cell stain kit (blue or violet) (ThermoFisher). The following anti-mouse antibodies were used for staining: CD4-FITC (clone: GK1.5), CD25-PE (PC61.5), Foxp3-PE-Cy7 (FJK-6s), CD11c-FITC (N418), CD11b-PE (M1/70), CD45-APC (30-F11), IFN-γ-PE (XMG1.2), and IL-17A-APC (eBio17B7) (All purchased from eBioscience). CD3-Pacific blue (17A2) was purchase from BioLegend (San Diego, Calif.). For CD45/CD11b/CD11c detection, the cells were subjected for analysis without fixation. For regulatory T cell (CD3/CD4/CD25/Foxp3) staining, after staining with the cell surface markers, the cells were fixed and permeabilized with the Foxp3 staining buffer kit (eBioscience) and stained with Foxp3-PE-Cy7. For Th1 and Th17 detection (CD3/CD4/IFN-γ/IL-17), single cell suspensions were stimulated for 4 hours with 50 ng/mL phorbal myristate acetate (PMA) and 500 ng/ml ionomysin in culture media (10% FBS, RPMI1640, penicillin, streptomycin, β-mercaptoethanol) in the presence of GolgiPlug™ (BD Biosciences). The cells were stained with CD3-PB, CD4-FITC, and Live/Dead blue then fixed and permeabilized using an intracellular fixation and permeabilization buffer set (eBioscience). The cells were next stained with IFN-γ-PE and IL-17-APC. Flow cytometric data were acquired on a LSR II (BD Biosciences). Acquired data was analyzed using FlowJo 10.1 ( ).

### Lymphocyte Proliferation Assay

The draining LN and spleens were collected and the cells were suspended at 2×10⁵ per 200 μL medium in 96-well flat-bottom plates. Triplicated cells were cultured in the presence of 10 μg/mL IRBP-p, 1 μg/mL Concanavalin A (Con A; Sigma-Aldrich), or medium alone. After incubation for three days, 100 μl of supernatant in the culture medium was collected. Cell proliferation during the last 4 hours of 72 hours cultures was measured by modified MTT assay using Cell Counting Kit-8 (Sigma-Aldrich).

### Microglia Depletion

Microglia depletion was performed using Cx3cr1^{CreER}×B6-iDTR (TG) mice or chow containing a small molecule CSFR1 inhibitor (Plexxikon Inc, Berkely, Calif., USA). To generate TG mice, Cx3cr1^{CreER} mice, which express Cre-ER fusion protein from endogenous CX3CR1 promoter enhance elements, were crossed with B6-iDTR mice, which contain a flox-STOP-flox diphtheria toxin receptor (DTR) in the ROSA26 locus. In this TG mice system, the Cre recombinase activation under the control of the Cx3cr1 promoter can be induced by tamoxifen, which leads to the surface expression of DTR on CX3CR1-expressing cells. The activation of Cre recombinase was induced by 5 consecutive days of tamoxifen administration via eye drops (10 μl/drop of 5 mg/ml in corn oil) three times a day [40] at 6 weeks old. Then diphtheria toxin (DTX) (Sigma-Aldrich) was administered into the anterior chamber (a.c.) (25 ng in 1 μl of saline) to deplete CX3CR1-expressing [43]. The control mice were given saline (a.c.). For microglia depletion using a small molecule CSFR1 inhibitor, mice were fed the control chow (AIN-76) or the chow containing 1200 p.p.m of the CSF1R inhibitor PLX 5622 one week prior to RD creation. No obvious behavioral or health problems were observed as a result of the a small molecule CSFR1 inhibitor supplemented diet.

### Statistical Analysis

Data are presented as the mean±standard error of the mean (SEM). Differences between two groups were analyzed using unpaired t-test or Mann-Whitney test. Multiple-group comparison was performed by one-way ANOVA followed by Tukey's or Dunnet's multiple comparison test. All of the statistic analysis was performed using graphing software (Prism 6, GraphPad Software, Inc., La Jolla, Calif., USA). The significance levels are marked *P<0.05; **P<0.01; ***P<0.001; ****P<0.0001 in figures.

### Example 1. Microglia Depletion with a Small Molecule CSFR1 Inhibitor Suppresses Uveitis but does not Suppress the Systemic Immune Response Against the Autoantigen (IRBP-p)

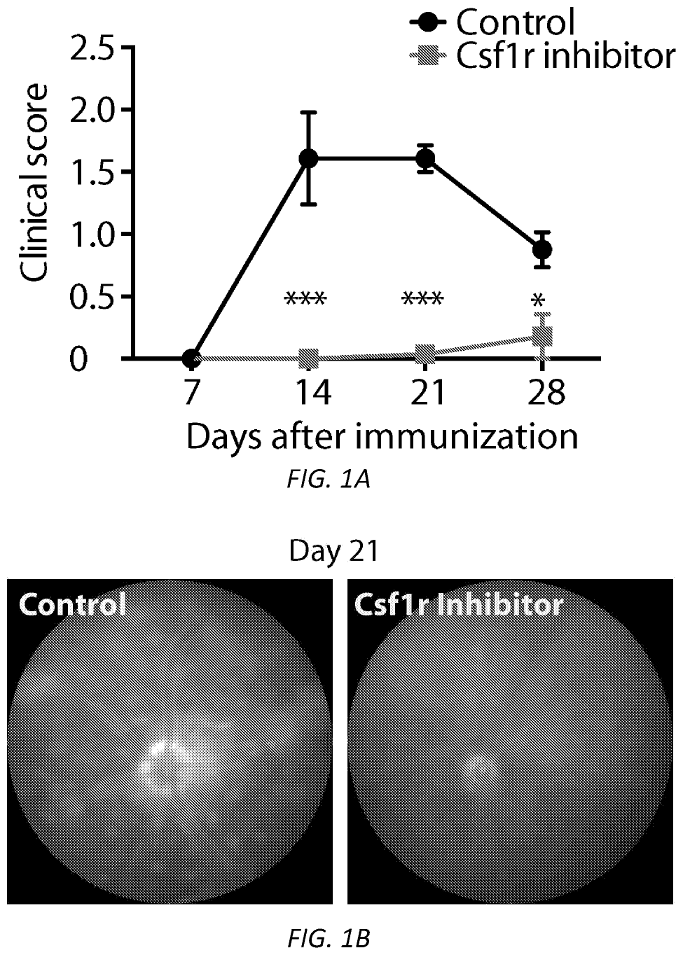

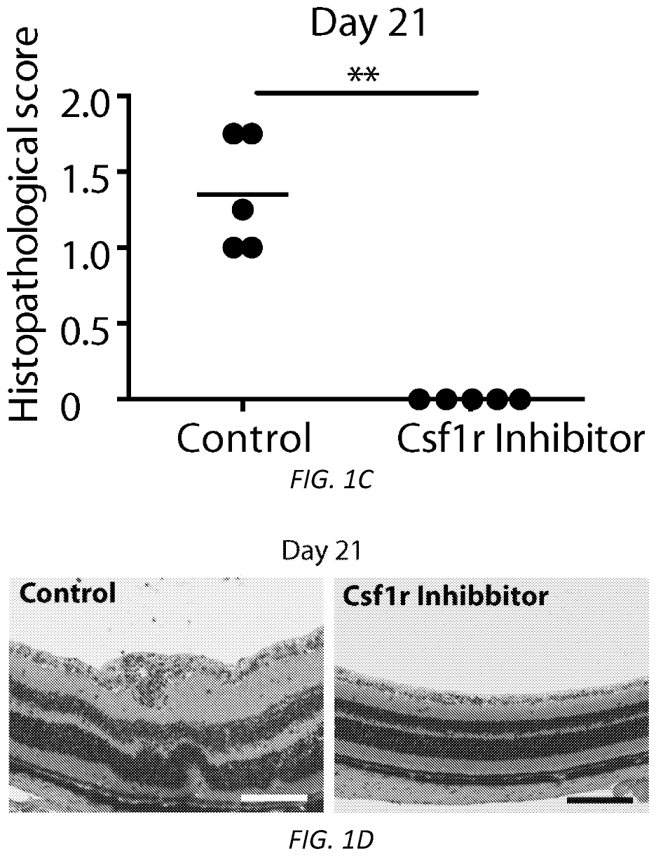

To define the role of retinal microglia in EAU, we first determined if microglia depletion affected EAU disease progression. To accomplish this, we utilized a Csf1R antagonist (a small molecule CSFR1 inhibitor), which has been shown to selectively induce cell death in microglia [29, 31, 32]. a small molecule CSFR1 inhibitor rapidly depletes retinal microglia within 7 days of beginning treatment. Dietary chow containing a small molecule CSFR1 inhibitor (1200 ppm) or a matched control diet was started 7 days prior to EAU induction to ensure complete loss of retinal microglia in a small molecule CSFR1 inhibitor-treated animals. EAU was induced by active immunization of the uveitogenic antigen IRBP-p and clinical assessment of EAU pathology was followed 7, 14, 21 and 28 days after EAU induction. Microglial depletion completely suppressed the development of EAU through day 21 (p<0.001). Only one animal in the a small molecule CSFR1 inhibitor group developed mild EAU by day 28, although 100% (7/7) of the control animals developed EAU by day 21 (FIG. 1A, B). Further, the histological score from mice with and without microglial depletion on day 21 confirmed that depletion of retinal microglia suppressed EAU induction (p<0.01) (FIG. 1C, D). Cumulatively, these data demonstrate that microglial depletion suppresses EAU pathology, indicating that microglia play a vital role in EAU pathogenesis.

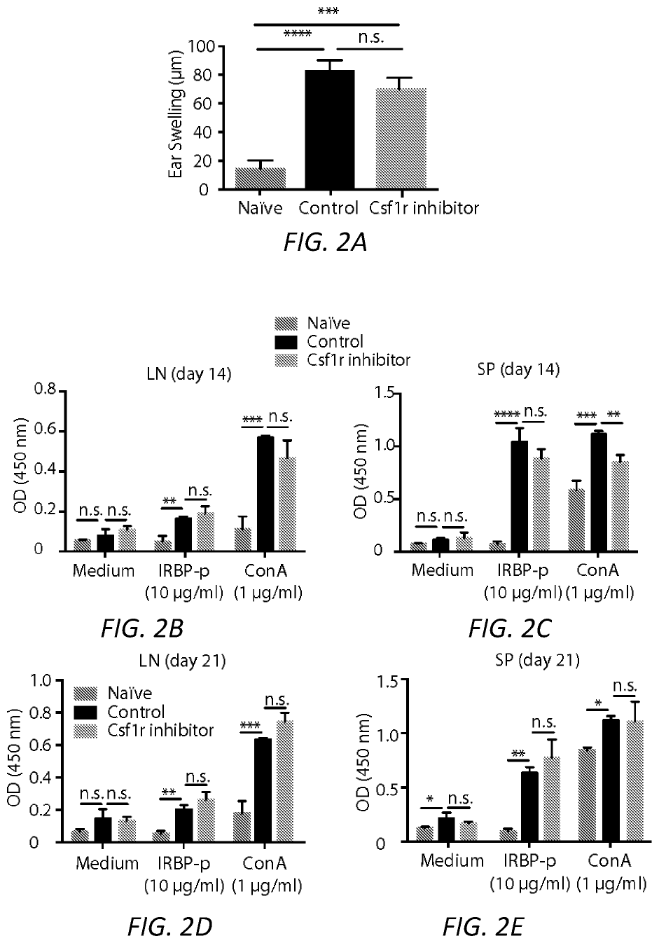

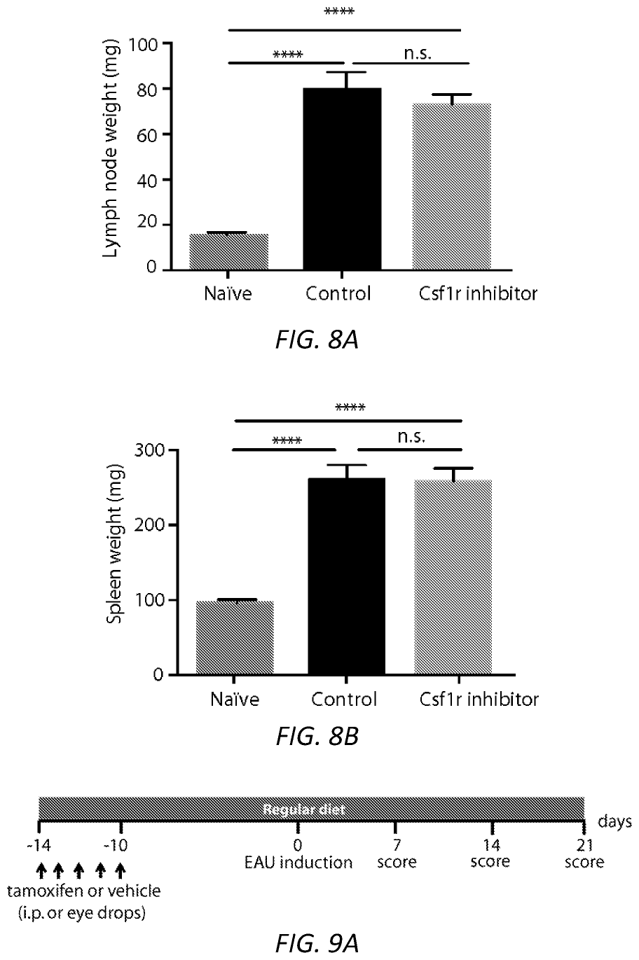

Our data and prior findings demonstrated that retinal microglia require Csf1R for survival. However, Csf1R is also expressed on systemic macrophages/monocytes and we therefore cannot exclude the possibility that the Csf1R antagonist systemically suppresses EAU, although previous studies have indicated that the Csf1R antagonist a small molecule CSFR1 inhibitor has a minimal effect on circulating systemic immune cells [31, 32]. Nevertheless, we evaluated potential systemic effects of a small molecule CSFR1 inhibitor in our EAU model. We first examined the systemic immune response against the immunized peptide IRBP-p by measuring delayed hypersensitivity (DH) as determined by ear swelling and lymphocyte proliferation in naïve animals, control and microglial-depleted animals with EAU. We found that a small molecule CSFR1 inhibitor did not affect the DH response or lymphocyte proliferation in IRBP-immunized animals (FIG. 2A-E). We also measured lymph node (LN) and spleen weight at the experimental endpoint of 21 days, and found that neither parameter was affected by a small molecule CSFR1 inhibitor (FIG. 8A, B). These data suggest that microglial depletion by a small molecule CSFR1 inhibitor does not affect systemic immune lymphocyte reactivity against IRBP-p in response to EAU induction, and that a small molecule CSFR1 inhibitor suppression of EAU is likely due to microglial depletion.

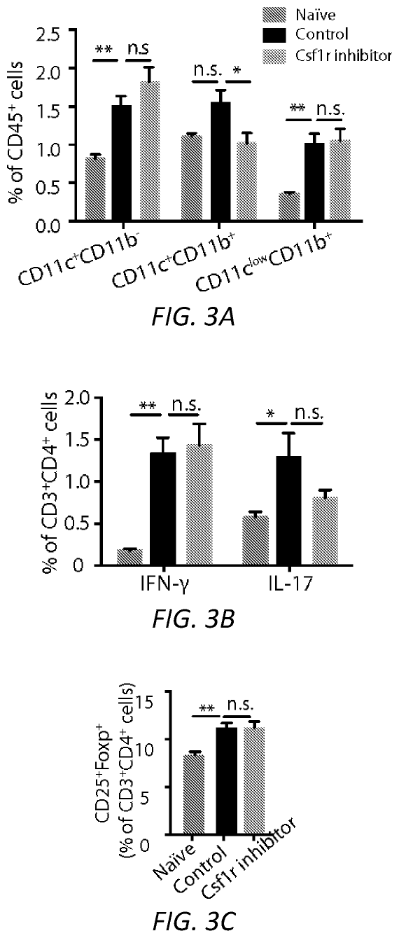

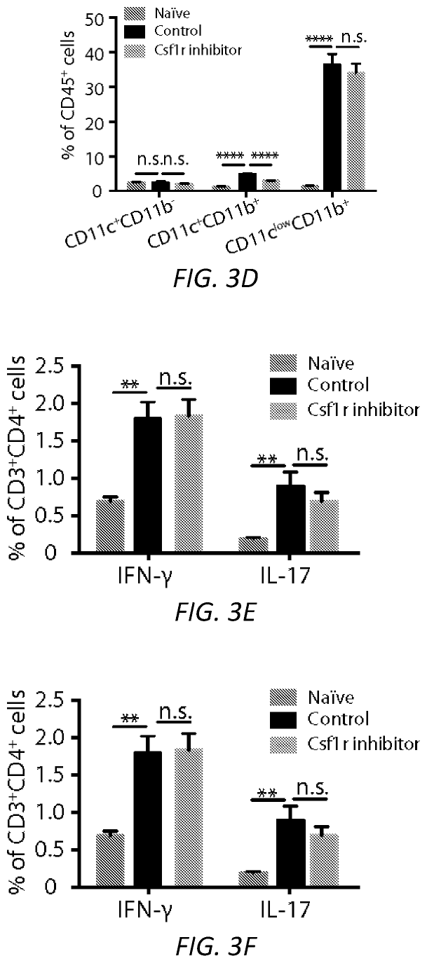

To further evaluate a small molecule CSFR1 inhibitor-mediated alterations of systemic immune cell populations in mice with EAU, we examined key regulatory cytokines and cell marker expression in LN cells and splenic cells using flow cytometry. We found that in both LNs and splenic cells, CD11c⁺CD11b⁺ cells, a dendritic cell population which is essential in antigen presentation against CD4⁺ T cells in EAU [33, 34], were decreased in the a small molecule CSFR1 inhibitor-treated group compared to control-fed animals on day 14 of EAU (FIG. 3A, D). Contrastingly, a small molecule CSFR1 inhibitor did not significantly change the frequency of CD3⁺CD4⁺ T cells positive for IFN-γ⁺ or IL-17⁺ on day 14, which are two major pathogenic cytokines in EAU (FIG. 3B, E) [35]. In addition, a small molecule CSFR1 inhibitor did not increase the frequency of regulatory T cells (CD4⁺CD25⁺Foxp3⁺), which are known to suppress EAU (FIG. 3C, E) [36]. These data demonstrate that although a small molecule CSFR1 inhibitor suppresses the population of CD11c⁺CD11b⁺ cells, a small molecule CSFR1 inhibitor does not largely affect systemic cytokine production and regulatory T cells in EAU. Cumulatively, these data suggest that a small molecule CSFR1 inhibitor does not affect cell priming in EAU. Furthermore, our DH studies (FIG. 2A-E) suggest that the systemic immune system is significantly primed against IRBP-p.

### Example 2. The Csf1R Antagonist, a Small Molecule CSFR1 Inhibitor, does not Affect Cell Priming in EAU

To further evaluate if a small molecule CSFR1 inhibitor affects cell priming in EAU, we utilized the adoptive transfer model of EAU. In the early phase of EAU induction, which occurs prior to the development of ocular inflammation, systemic immune cells are primed by active immunization. The later phase of EAU induction is regarded as the effector phase, wherein activated cells infiltrate the retina and induce inflammation [37, 38]. In the adoptive transfer model of EAU, autoreactive cells are transferred from donor EAU-induced mice to naïve mice. The transferred cells induce EAU in recipient mice, bypassing the induction phase. Thus, by transferring IRBP-p reactive immune cells from donor animals to recipient mice with a small molecule CSFR1 inhibitor microglial suppression, we are able to assess the contribution of microglia on EAU suppression, and exclude the effect of CSF1R antagonism in the cell priming stage.

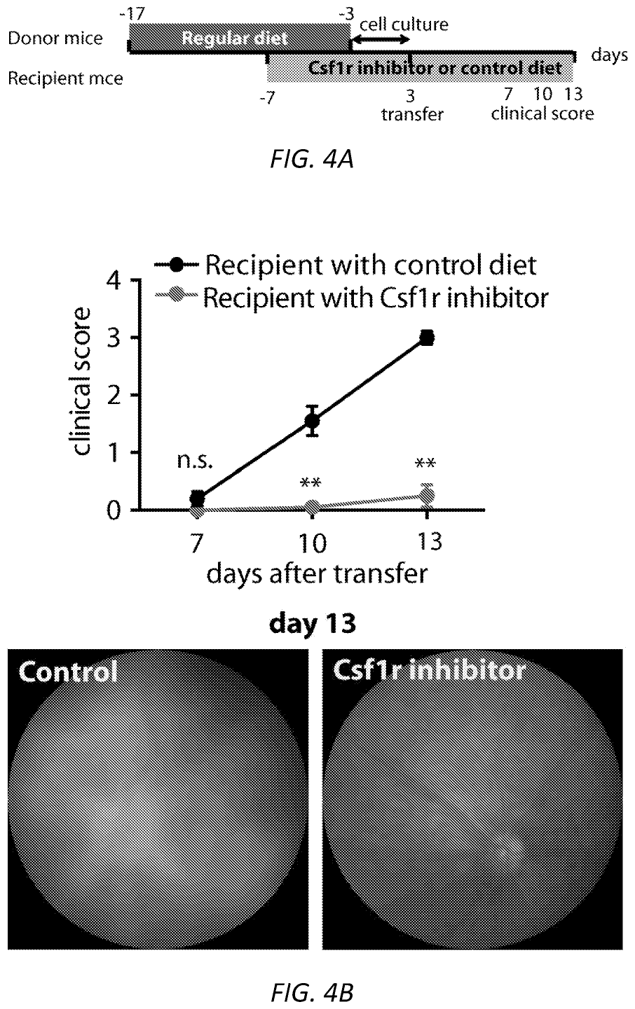

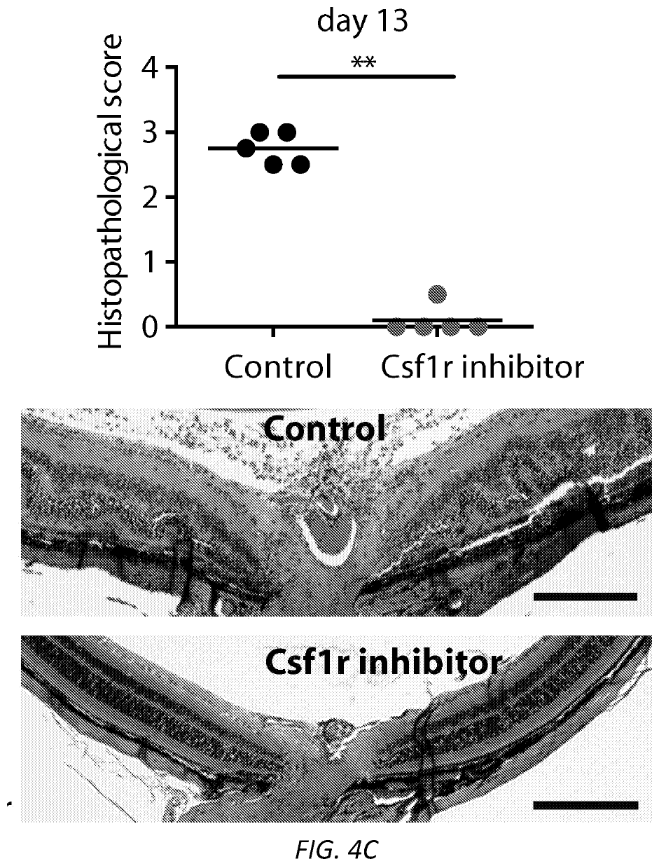

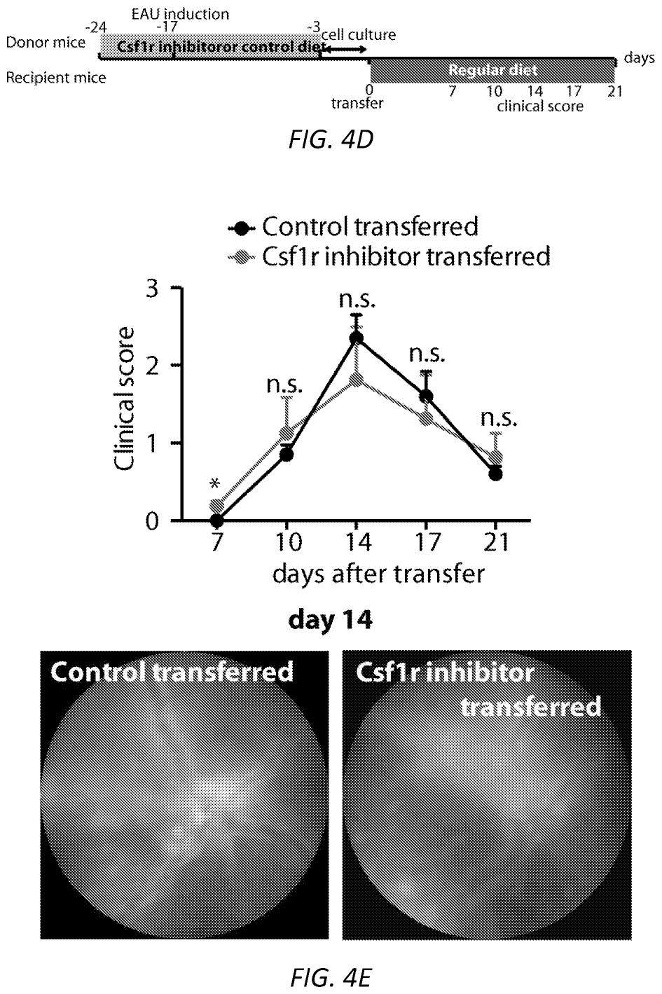

When naïve recipient animals fed a control diet received primed cells transferred from donor mice with EAU, significant inflammation characteristic of EAU was induced in recipient animals (FIG. 4A-C). Conversely, the EAU response was suppressed in naïve a small molecule CSFR1 inhibitor-treated recipient animals (FIG. 4A-C). These results indicate that the EAU suppressive effect of a small molecule CSFR1 inhibitor is elicited without suppressing the systemic cell priming, and is largely due to loss of retinal microglia.

Conversely, we treated donor animals with a small molecule CSFR1 inhibitor or control diet 7 days prior to EAU induction, and transferred primed cells to recipient naïve animals on a regular diet. The recipient naïve mice from both donors had a significant induction of EAU, and a small molecule CSFR1 inhibitor treatment in donor animals did not affect the severity of EAU (FIG. 4D, E). This result further confirms that any effects of a small molecule CSFR1 inhibitor on cell priming or systemic immune cells are not operative in EAU, and that a small molecule CSFR1 inhibitor suppression of EAU is due to retinal microglial depletion.

### Example 3. Depleting Retinal Microglia with Local CX3CR1 Ablation Suppresses EAU

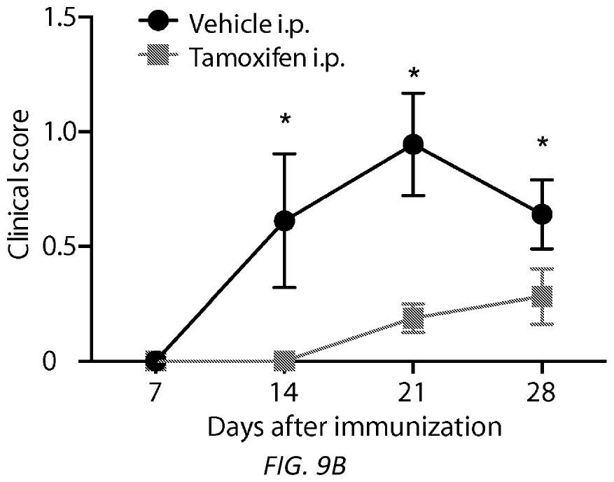

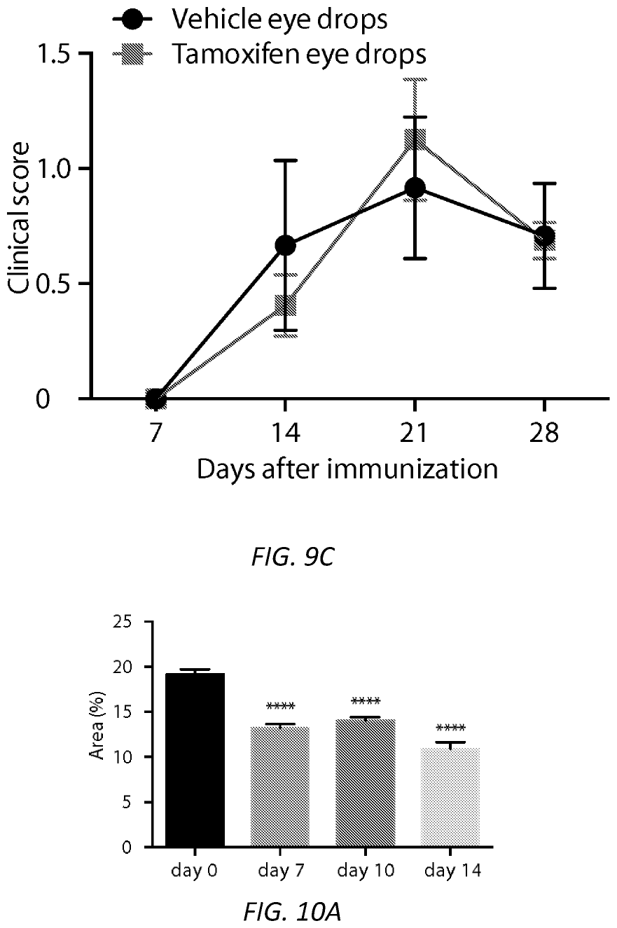

We next depleted retinal microglia utilizing a transgenic (TG) mouse approach. Cx3cr1^{CreER} mice were crossed to B6-iDTR mice (Cx3cr1^{CreER}×B6-iDTR). In subsequent offspring, Cre recombinase activation under the control of the Cx3cr1 promoter can be induced by tamoxifen, leading to expression of the human diphtheria toxin receptor on CX3CR1-expressing cells. Under normal conditions, most CX3CR1-positive cells in the retina are microglia, so tamoxifen would induce diphtheria toxin receptor expression in predominately microglia. In this model system, cells expressing diphtheria toxin receptor can be depleted by administration of diphtheria toxin (DTX), thus depleting microglia with ocular administration of DTX [39]. We induced Cre recombinase activation for 5 consecutive days with tamoxifen administration via eye drops (3 times per day) in mice beginning at 6 weeks of age [40]. Topical administration of tamoxifen was used because tamoxifen has known immuno-suppressive effects in animal models of autoimmune diseases [41, 42]. Accordingly, when tamoxifen alone was given systemically via i.p. injection in Cx3cr1^{CreER}×B6-iDTR mice, EAU was significantly suppressed, whereas topical administration of tamoxifen via eye drops did not significantly affect EAU severity (FIG. 9A-C).

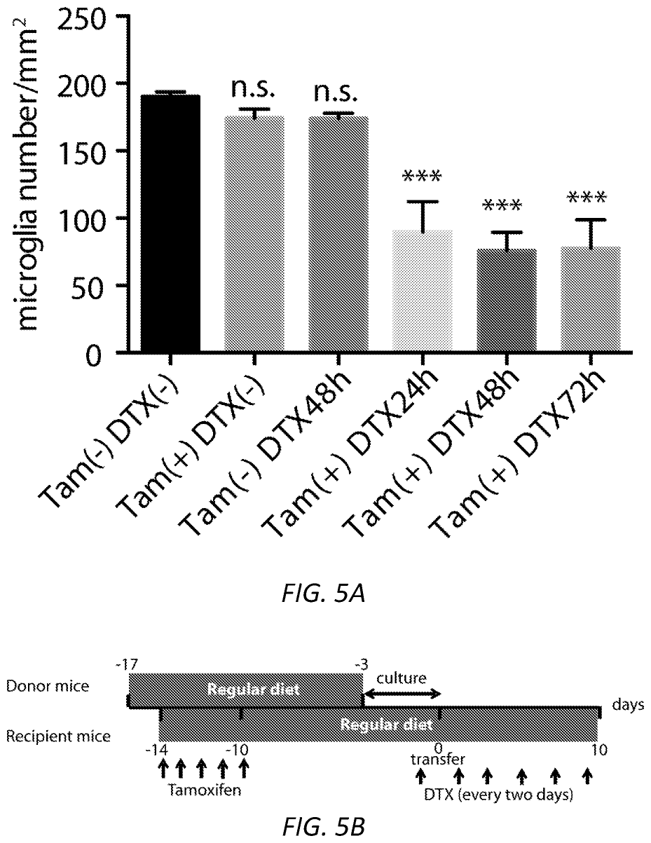

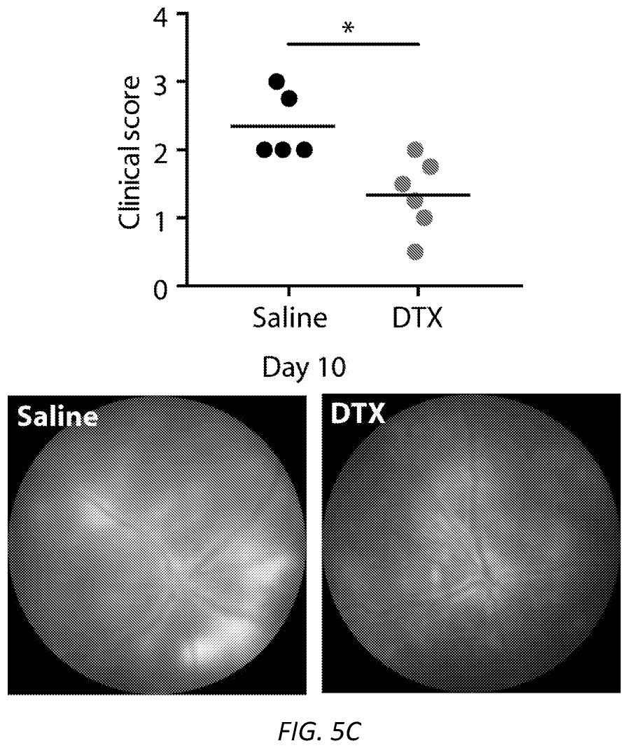

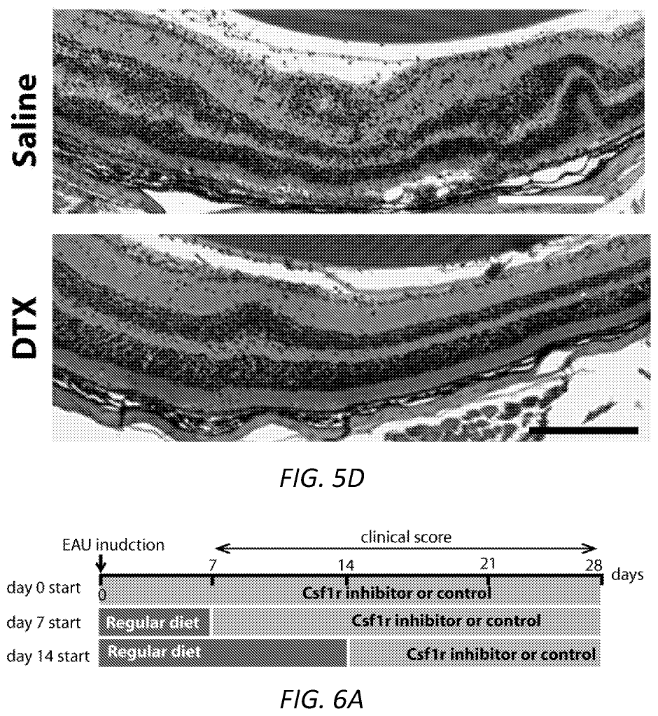

Retinal microglia were depleted by introducing DTX via the anterior chamber (a.c.) [43] in tamoxifen-treated TG mice. 60% of retinal microglia were depleted in 48 hours with this microglia elimination approach (FIG. 5A). Accordingly, we started DTX (a.c.) administration on day −1, and EAU was adoptively induced on day 0. The adoptive transfer model of EAU was utilized in order to minimize the number of DTX injections needed, as inflammation develops more rapidly in the adoptive transfer model than in the active immunization model [37]. DTX a.c. administration was repeated every two days until day 9 and the eyes were evaluated clinically and histopathologically on day 10 (FIG. 5B). EAU was significantly suppressed in microglia-depleted mice (FIG. 5C, D). EAU suppression in the transgenic model of microglial depletion was not as significant as observed with the Csf1R antagonist, likely due to the degree of microglia depletion in both approaches (60% depletion in the TG system versus 100% depletion in a small molecule CSFR1 inhibitor-treated mice). This study further confirmed that microglia direct the immune response and pathology in autoimmune uveitis.

### Example 4. Suppression of EAU Through Microglial Depletion is Time Dependent

EAU is significantly suppressed by administration of Csf1R antagonist a small molecule CSFR1 inhibitor prior to EAU induction (FIG. 1A-D), indicating that retinal microglia play a vital role in directing the autoimmune response to the retina. However, it was still unclear if retinal microglia play a role in propagating the immune response after EAU had been induced and immune cells had gained entry into the retina. To begin to address this, we administered a small molecule CSFR1 inhibitor at several time points before and after EAU induction and examined the severity of EAU.

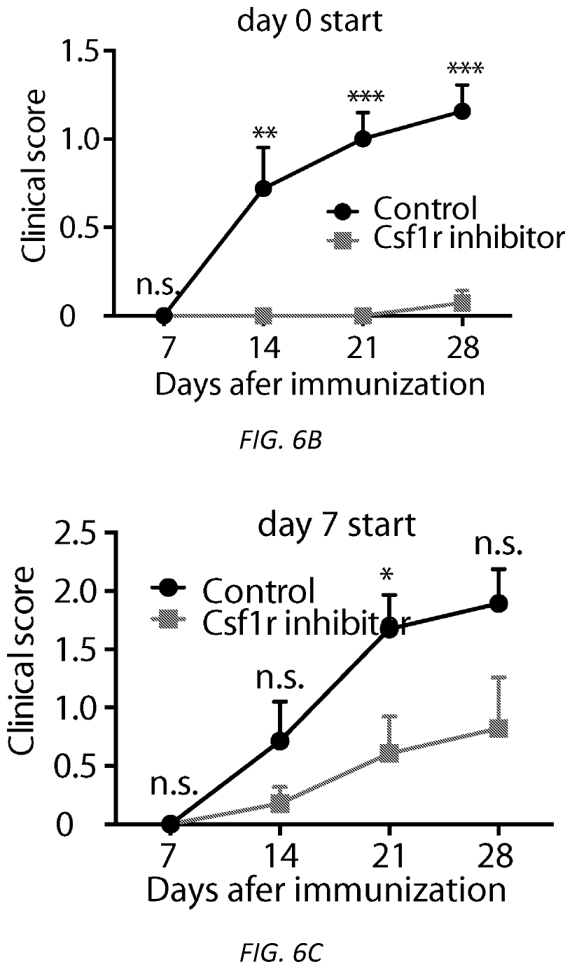

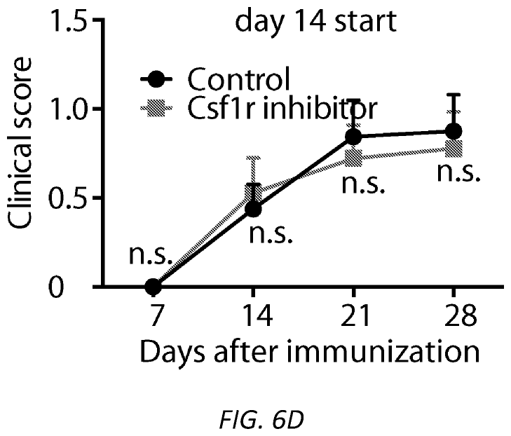

In our EAU model, inflammation was not observed until day 7 and was first observed around 10 days after immunization, indicating that autoimmune cell entry occurs between days 7 and 10. We started a small molecule CSFR1 inhibitor on the day of EAU induction (day 0), day 7, and day 14 (FIG. 6A). When a small molecule CSFR1 inhibitor was started on the day of EAU induction (day 0), EAU was effectively suppressed until 28 days after EAU induction (FIG. 6B). Because a small molecule CSFR1 inhibitor depletes retinal microglia in 7 days, microglia should have been depleted before the development of EAU in this group. When a small molecule CSFR1 inhibitor was started on day 7, EAU was partially suppressed (FIG. 6C). In this group, a small population of microglia was present at the beginning of EAU at day 10, and microglia depletion was completed during the development of EAU. When a small molecule CSFR1 inhibitor was started on day 14, the severity of EAU in a small molecule CSFR1 inhibitor fed mice was comparable to that of control mice (FIG. 6D). In this group, a full population microglia was present during the development of EAU, and the population was depleted after the development of EAU.

These results point to a central role for microglia in the development of EAU. However, the a small molecule CSFR1 inhibitor time course suggests that microglia are not as relevant once infiltrating immune cells have entered the retina, following the initial induction of EAU. Taken together, these data suggest that microglial depletion only suppresses EAU when microglia have been successfully depleted prior to immune cell infiltration and before the advance stages of EAU have developed. These data strongly suggest that microglia have important roles in induction of EAU.

### Example 5. Microglia are Localized in the Inner Retina During EAU Disease Induction

To characterize microglial activation in response to EAU, we assessed how microglia change their morphology, number and location during disease induction. Whole mount retinas from naïve mice (day 0) and EAU mice on day 7, 10, and 14 were stained with P2ry12 (a microglia-specific marker) and lectin (an endothelial marker for vessel staining), and the number and morphology of microglia were then evaluated using confocal microscopy.

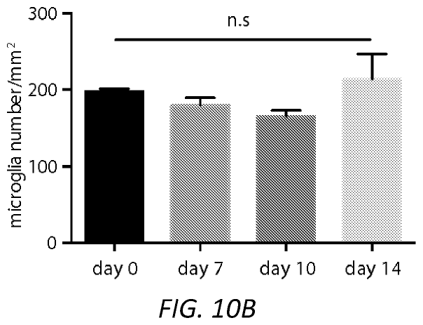

In this model of EAU, immune cell infiltration into the retinal microenvironment begins between days 7 and 10 post disease induction. We observed that microglia progress from a highly-ramified appearance into a more activated amoeboid shape (FIG. 10A). Previous reports have indicated that P2ry12 is downregulated in certain disease conditions. However, we did not observe any significant changes in microglial number, as indicated by P2ry12 staining through EAU day 14 (FIG. 10B). Retinal microglia were located proximal to and closely associated with the retinal vascular plexus, and upon disease induction these cells remained within this vascularized region. Of interest, microglia appeared to become more closely associated with retinal vessels during EAU disease progression. These results demonstrate that microglia become activated by day 7, prior to development of clinically apparent EAU.

### Example 6. Loss of Leukocyte Recruitment in Microglia-depleted Retinas During EAU

In the induction of EAU, leukocyte trafficking (rolling and infiltration) concurrent with upregulation of adhesion molecules (such as ICAM-1 and VCAM-1) in the retinal vessels drives EAU pathogenesis [10]. Because microglia are closely associated with the microvasculature and contribute to EAU induction, we hypothesized that microglia may regulate leukocyte trafficking to the retina. To address this hypothesis, we examined the number of adherent cells and expression of key adhesion molecules in the retina in response to EAU disease induction.

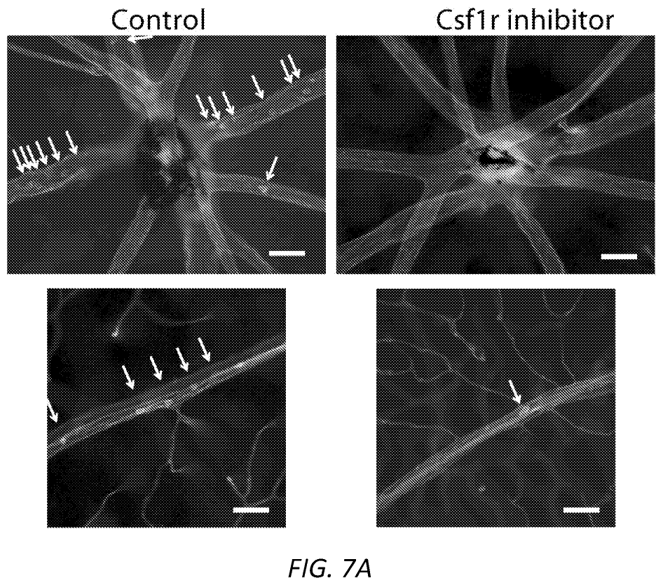

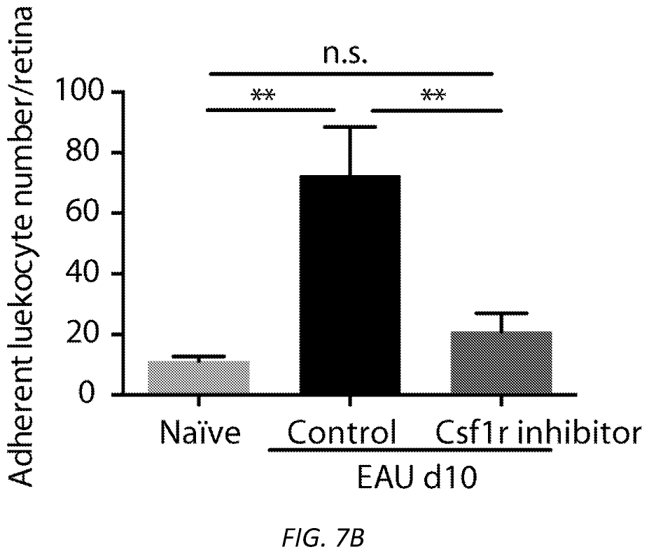

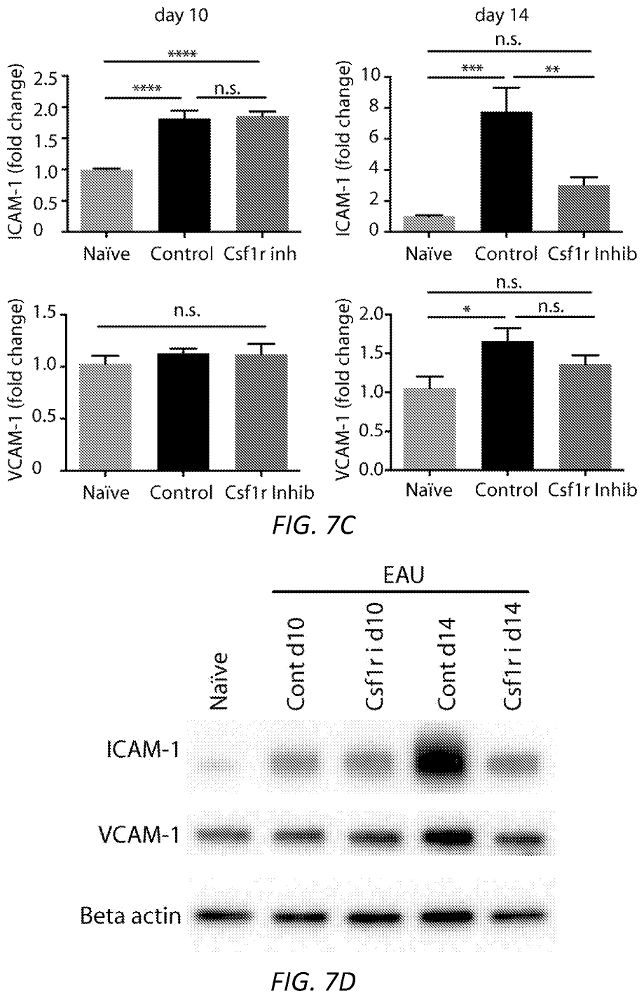

We found that a small molecule CSFR1 inhibitor-treated EAU mice had significantly fewer adherent cells than control EAU mice, and that levels of adherent cells were in fact similar between a small molecule CSFR1 inhibitor-treated EAU mice and naïve mice on day 10 (FIG. 7A). However, on day 10 retinal ICAM-1 protein expression was significantly higher in a small molecule CSFR1 inhibitor-fed EAU mice than in the naïve retina (P<0.01), and was in fact similar to that of control-fed EAU mice (FIG. 7B). On day 14, ICAM-1 expression in control-fed EAU mice largely increased. Retinal VCAM-1 expression was upregulated in control-fed EAU retinas on day 14, and was unaffected by a small molecule CSFR1 inhibitor treatment (FIG. 7C). Immunohistochemistry (IHC) of ICAM-1 and VCAM-1 in retinas from control- and a small molecule CSFR1 inhibitor-fed mice on day 10 of EAU revealed that ICAM-1 and VCAM-1 are upregulated predominately in the retinal vessels.

In summary, depletion of retinal microglia with a small molecule CSFR1 inhibitor significantly decreases leukocyte adhesion in EAU, although expression of adhesion molecules is unaffected. Because lymphocytes from a small molecule CSFR1 inhibitor-fed EAU donor mice were equally potent to those from control-fed EAU donor mice in the adoptive transfer model of EAU, it is unlikely that downregulation of ligands against adhesion molecules, such as lymphocyte function-associated antigen (LFA)-1, is the main cause for a decrease in cell adhesion. These results indicate that leukocyte adhesion is interrupted the in microglia-depleted retina, although blood vessel expression of adhesion molecules and adhesion molecule ligand in trafficking leukocytes is unaffected. This suggests that microglia enhance cell adhesion independently of cell adhesion marker expression or activation.

### Example 7. Microglia Directly Interact with Adhesive Immune Cells in the Induction Phase of EAU

We next determined if microglia have direct contact with adherent leukocytes in the early phase of EAU. EAU was induced in C57BL/6 mice on a regular diet, and the retinas were collected for IHC on day 7 and 10. The animals were perfused with 20 ml of PBS before sacrifice to wash out non-adherent cells in the vessels. Antibodies against MHC-class II, CD11b, or CD4/CD8 were used to label leukocytes, with labeling of microglia and blood vessels using P2ry12 and lectin, respectively. Under the direct observation with confocal microscopy, microglia located close to intravessel leukocytes were chosen, and z-stack images of those microglia were taken. The z-stack and 3D-constructed images were created to examine the three-dimensional association among microglia, leukocytes, and vessel walls. We observed direct association of microglia and adherent leukocytes through microglial processes in EAU (FIG. 8). Microglial interaction with MHC-class II⁺ cells was first observed at day 7 of EAU, particularly on day 10 (FIG. 8A). Microglia did not express MCH-class II on day 7 and 10 of EAU (FIG. 8A). Intravascular CD11b⁺ cells and CD4/CD8⁺ cells also directly interacted with microglia (FIG. 8B). 3D-constructed images demonstrated that microglia have direct contact with these leukocytes, which are located on the intravascular wall.

Based on these observations, we suggest that microglia play a critical role in induction of EAU by enhancing and stabilizing cell adhesion of rolling leukocytes through direct contact with leukocytes. Some of these leukocytes might eventually infiltrate into the retina and trigger larger inflammatory cell recruitment in later time points.

### REFERENCES

1. Goto H, Mochizuki M, Yamaki K, Kotake S, Usui M, Ohno S: Epidemiological survey of intraocular inflammation in Japan. Japanese journal of ophthalmology 2007, 51 (1):41-44.
2. Durrani O M, Tehrani N N, Marr J E, Moradi P, Stavrou P, Murray P I: Degree, duration, and causes of visual loss in uveitis. The British journal of ophthalmology 2004, 88 (9):1159-1162.
3. Lee R W, Nicholson L B, Sen H N, Chan C C, Wei L, Nussenblatt R B, Dick A D: Autoimmune and autoinflammatory mechanisms in uveitis. Seminars in immunopathology 2014, 36 (5):581-594.
4. Caspi R R, Silver P B, Luger D, Tang J, Cortes L M, Pennesi G, Mattapallil M J, Chan C C: Mouse models of experimental autoimmune uveitis. Ophthalmic Res 2008, 40 (3-4):169-174.
5. Rizzo L V, Silver P, Wiggert B, Hakim F, Gazzinelli R T, Chan C C, Caspi R R: Establishment and characterization of a murine CD4+ T cell line and clone that induce experimental autoimmune uveoretinitis in B10.A mice. J Immunol 1996, 156 (4):1654-1660.
6. Sanui H, Redmond T M, Kotake S, Wiggert B, Hu L H, Margalit H, Berzofsky J A, Chader G J, Gery I: Identification of an immunodominant and highly immunopathogenic determinant in the retinal interphotoreceptor retinoid-binding protein (IRBP). J Exp Med 1989, 169 (6):1947-1960.
7. Lipski D A, Dewispelaere R, Foucart V, Caspers L E, Defrance M, Bruyns C, Willermain F: MHC class II expression and potential antigen-presenting cells in the retina during experimental autoimmune uveitis. J Neuroinflammation 2017, 14 (1):136.
8. Gregerson D S, Sam T N, McPherson S W: The antigen-presenting activity of fresh, adult parenchymal microglia and perivascular cells from retina. J Immunol 2004, 172 (11):6587-6597.
9. Chan C C, Caspi R R, Roberge F G, Nussenblatt R B: Dynamics of experimental autoimmune uveoretinitis induced by adoptive transfer of S-antigen-specific T cell line. Invest Ophthalmol Vis Sci 1988, 29 (3):411-418.
10. Xu H, Forrester J V, Liversidge J, Crane I J: Leukocyte trafficking in experimental autoimmune uveitis: breakdown of blood-retinal barrier and upregulation of cellular adhesion molecules. Invest Ophthalmol Vis Sci 2003, 44 (1):226-234.
11. Dewispelaere R, Lipski D, Foucart V, Bruyns C, Frere A, Caspers L, Willermain F: ICAM-1 and VCAM-1 are differentially expressed on blood-retinal barrier cells during experimental autoimmune uveitis. Exp Eye Res 2015, 137:94-102.
12. Ginhoux F, Greter M, Leboeuf M, Nandi S, See P, Gokhan S, Mehler M F, Conway S J, Ng L G, Stanley E R et al: Fate mapping analysis reveals that adult microglia derive from primitive macrophages. Science 2010, 330 (6005):841-845.
13. Lam T T, Kwong J M, Tso M O: Early glial responses after acute elevated intraocular pressure in rats. Investigative ophthalmology & visual science 2003, 44 (2):638-645.
14. Gupta N, Brown K E, Milam A H: Activated microglia in human retinitis pigmentosa, late-onset retinal degeneration, and age-related macular degeneration. Experimental eye research 2003, 76 (4):463-471.
15. Penfold P L, Madigan M C, Gillies M C, Provis J M: Immunological and aetiological aspects of macular degeneration. Prog Retin Eye Res 2001, 20 (3):385-414.
16. Zeng H Y, Green W R, Tso M O: Microglial activation in human diabetic retinopathy. Archives of ophthalmology 2008, 126 (2):227-232.
17. Zhao L, Ma W, Fariss R N, Wong W T: Retinal vascular repair and neovascularization are not dependent on CX3CR1 signaling in a model of ischemic retinopathy. Experimental eye research 2009, 88 (6):1004-1013.
18. Connor K M, SanGiovanni J P, Lofqvist C, Aderman C M, Chen J, Higuchi A, Hong S, Pravda E A, Majchrzak S, Carper D et al: Increased dietary intake of omega-3-polyunsaturated fatty acids reduces pathological retinal angiogenesis. Nat Med 2007, 13 (7):868-873.
19. Fischer A J, Zelinka C, Milani-Nejad N: Reactive retinal microglia, neuronal survival, and the formation of retinal folds and detachments. Glia 2015, 63 (2):313-327.
20. Lewis G P, Sethi C S, Carter K M, Charteris D G, Fisher S K: Microglial cell activation following retinal detachment: a comparison between species. Molecular vision 2005, 11:491-500.
21. Nakazawa T, Hisatomi T, Nakazawa C, Noda K, Maruyama K, She H, Matsubara A, Miyahara S, Nakao S, Yin Y et al: Monocyte chemoattractant protein 1 mediates retinal detachment-induced photoreceptor apoptosis. Proceedings of the National Academy of Sciences of the United States of America 2007, 104 (7):2425-2430.
22. Dagkalis A, Wallace C, Hing B, Liversidge J, Crane I J: CX3CR1-deficiency is associated with increased severity of disease in experimental autoimmune uveitis. Immunology 2009, 128 (1):25-33.
23. Couturier A, Bousquet E, Zhao M, Naud M C, Klein C, Jonet L, Tadayoni R, de Kozak Y, Behar-Cohen F: Anti-vascular endothelial growth factor acts on retinal microglia/macrophage activation in a rat model of ocular inflammation. Mol Vis 2014, 20:908-920.
24. Karlstetter M, Scholz R, Rutar M, Wong W T, Provis J M, Langmann T: Retinal microglia: Just bystander or target for therapy? Prog Retin Eye Res 2014.
25. Karlstetter M, Ebert S, Langmann T: Microglia in the healthy and degenerating retina: insights from novel mouse models. Immunobiology 2010, 215 (9-10):685-691.
26. Gullapalli V K, Zhang J, Pararajasegaram G, Rao N A: Hematopoietically derived retinal perivascular microglia initiate uveoretinitis in experimental autoimmune uveitis. Graefes Arch Clin Exp Ophthalmol 2000, 238 (4):319-325.
27. Salter M W, Beggs S: Sublime microglia: expanding roles for the guardians of the CNS. Cell 2014, 158 (1):15-24.
28. Butovsky O, Jedrychowski M P, Moore C S, Cialic R, Lanser A J, Gabriely G, Koeglsperger T, Dake B, Wu P M, Doykan C E et al: Identification of a unique TGF-beta-dependent molecular and functional signature in microglia. Nat Neurosci 2014, 17 (1):131-143.
29. Ebneter A, Kokona D, Jovanovic J, Zinkernagel M S: Dramatic Effect of Oral CSF-1R Kinase Inhibitor on Retinal Microglia Revealed by In Vivo Scanning Laser Ophthalmoscopy. Transl Vis Sci Technol 2017, 6 (2):10.
30. Dagher N N, Najafi A R, Kayala K M, Elmore M R, White T E, Medeiros R, West B L, Green K N: Colony-stimulating factor 1 receptor inhibition prevents microglial plaque association and improves cognition in 3xTg-AD mice. J Neuroinflammation 2015, 12:139.
31. Valdearcos M, Robblee M M, Benjamin D I, Nomura D K, Xu A W, Koliwad S K: Microglia dictate the impact of saturated fat consumption on hypothalamic inflammation and neuronal function. Cell Rep 2014, 9 (6):2124-2138.
32. Hilla A M, Diekmann H, Fischer D: Microglia Are Irrelevant for Neuronal Degeneration and Axon Regeneration after Acute Injury. The Journal of neuroscience: the official journal of the Society for Neuroscience 2017, 37 (25):6113-6124.
33. Liyanage S E, Gardner P J, Ribeiro J, Cristante E, Sampson R D, Luhmann U F, Ali R R, Bainbridge J W: Flow cytometric analysis of inflammatory and resident myeloid populations in mouse ocular inflammatory models. Exp Eye Res 2016, 151:160-170.
34. Chen P, Denniston A K, Hirani S, Hannes S, Nussenblatt R B: Role of dendritic cell subsets in immunity and their contribution to noninfectious uveitis. Surv Ophthalmol 2015, 60 (3):242-249.
35. Luger D, Caspi R R: New perspectives on effector mechanisms in uveitis. Semin Immunopathol 2008, 30 (2):135-143.
36. Sun M, Yang P, Du L, Zhou H, Ren X, Kijlstra A: Contribution of CD4+CD25+ T cells to the regression phase of experimental autoimmune uveoretinitis. Invest Ophthalmol Vis Sci 2010, 51 (1):383-389.
37. Caspi R R: Experimental autoimmune uveoretinitis in the rat and mouse.

Current protocols in immunology 2003, Chapter 15: Unit 15 16.
38. Agarwal R K, Caspi R R: Rodent models of experimental autoimmune uveitis. Methods Mol Med 2004, 102:395-419.
39. Parkhurst C N, Yang G, Ninan I, Savas J N, Yates J R, 3rd, Lafaille J J, Hempstead B L, Littman D R, Gan W B: Microglia promote learning-dependent synapse formation through brain-derived neurotrophic factor. Cell 2013, 155 (7):1596-1609.
40. Boneva S K, Gross T R, Schlecht A, Schmitt S I, Sippl C, Jagle H, Volz C, Neueder A, Tamm E R, Braunger B M: Cre recombinase expression or topical tamoxifen treatment do not affect retinal structure and function, neuronal vulnerability or glial reactivity in the mouse eye. Neuroscience 2016, 325:188-201.
41. Bebo B F, Jr., Dehghani B, Foster S, Kurniawan A, Lopez F J, Sherman L S: Treatment with selective estrogen receptor modulators regulates myelin specific T-cells and suppresses experimental autoimmune encephalomyelitis. Glia 2009, 57 (7):777-790.
42. de Kozak Y, Andrieux K, Villarroya H, Klein C, Thillaye-Goldenberg B, Naud M C, Garcia E, Couvreur P: Intraocular injection of tamoxifen-loaded nanoparticles: a new treatment of experimental autoimmune uveoretinitis. European journal of immunology 2004, 34 (12):3702-3712.
43. McPherson S W, Heuss N D, Pierson M J, Gregerson D S: Retinal antigen-specific regulatory T cells protect against spontaneous and induced autoimmunity and require local dendritic cells. Journal of neuroinflammation 2014, 11:205.
44. Chitu V, Stanley E R: Colony-stimulating factor-1 in immunity and inflammation. Current opinion in immunology 2006, 18 (1):39-48.
45. Li J, Chen K, Zhu L, Pollard J W: Conditional deletion of the colony stimulating factor-1 receptor (c-fms proto-oncogene) in mice. Genesis 2006, 44 (7):328-335.
46. Valdearcos M, Robblee M M, Benjamin D I, Nomura D K, Xu A W, Koliwad S K: Microglia dictate the impact of saturated fat consumption on hypothalamic inflammation and neuronal function. Cell reports 2014, 9 (6):2124-2138.
47. Hou W, Wu Y, Sun S, Shi M, Sun Y, Yang C, Pei G, Gu Y, Zhong C, Sun B: Pertussis toxin enhances Th1 responses by stimulation of dendritic cells. J Immunol 2003, 170 (4):1728-1736.
48. Usui Y, Takeuchi M, Hattori T, Okunuki Y, Nagasawa K, Kezuka T, Okumura K, Yagita H, Akiba H, Goto H: Suppression of experimental autoimmune uveoretinitis by regulatory dendritic cells in mice. Arch Ophthalmol 2009, 127 (4):514-519.
49. Klaska I P, Muckersie E, Martin-Granados C, Christofi M, Forrester J V: Lipopolysaccharide-primed heterotolerant dendritic cells suppress experimental autoimmune uveoretinitis by multiple mechanisms. Immunology 2017, 150 (3):364-377.
50. Iadecola C: Neurovascular regulation in the normal brain and in Alzheimer's disease. Nat Rev Neurosci 2004, 5 (5):347-360.
51. Gardner T W, Davila J R: The neurovascular unit and the pathophysiologic basis of diabetic retinopathy. Graefes Arch Clin Exp Ophthalmol 2017, 255 (1):1-6.
52. Metea M R, Newman E A: Signalling within the neurovascular unit in the mammalian retina. Exp Physiol 2007, 92 (4):635-640.
53. Zlokovic B V: The blood-brain barrier in health and chronic neurodegenerative disorders. Neuron 2008, 57 (2):178-201.
54. Bell R D, Winkler E A, Sagare A P, Singh I, LaRue B, Deane R, Zlokovic B V: Pericytes control key neurovascular functions and neuronal phenotype in the adult brain and during brain aging. Neuron 2010, 68 (3):409-427.
55. da Fonseca A C, Matias D, Garcia C, Amaral R, Geraldo L H, Freitas C, Lima F R: The impact of microglial activation on blood-brain barrier in brain diseases. Front Cell Neurosci 2014, 8:362.
56. Sumi N, Nishioku T, Takata F, Matsumoto J, Watanabe T, Shuto H, Yamauchi A, Dohgu S, Kataoka Y: Lipopolysaccharide-activated microglia induce dysfunction of the blood-brain barrier in rat microvascular endothelial cells co-cultured with microglia. Cell Mol Neurobiol 2010, 30 (2):247-253.
57. Spanos J P, Hsu N J, Jacobs M: Microglia are crucial regulators of neuro-immunity during central nervous system tuberculosis. Front Cell Neurosci 2015, 9:182.
58. Prendergast R A, Iliff C E, Coskuncan N M, Caspi R R, Sartani G, Tarrant T K, Lutty G A, McLeod D S: T cell traffic and the inflammatory response in experimental autoimmune uveoretinitis. Invest Ophthalmol Vis Sci 1998, 39 (5):754-762.
59. Thurau S R, Mempel T R, Flugel A, Diedrichs-Mohring M, Krombach F, Kawakami N, Wildner G: The fate of autoreactive, GFP+ T cells in rat models of uveitis analyzed by intravital fluorescence microscopy and FACS. Int Immunol 2004, 16 (11):1573-1582.
60. Forrester J V, McMenamin P G, Holthouse I, Lumsden L, Liversidge J: Localization and characterization of major histocompatibility complex class II-positive cells in the posterior segment of the eye: implications for induction of autoimmune uveoretinitis. Investigative ophthalmology & visual science 1994, 35 (1):64-77.
61. Dick A D, Ford A L, Forrester J V, Sedgwick J D: Flow cytometric identification of a minority population of MHC class II positive cells in the normal rat retina distinct from CD45lowCD11b/c+CD4low parenchymal microglia. The British journal of ophthalmology 1995, 79 (9):834-840.
62. Zhang J X, Zha W S, Ye L P, Wang F, Wang H, Shen T, Wu C H, Zhu Q X: Complement C5a-C5aR interaction enhances MAPK signaling pathway activities to mediate renal injury in trichloroethylene sensitized BALB/c mice. Journal of applied toxicology: JAT 2016, 36 (2):271-284.
63. Thurau S R, Chan C C, Nussenblatt R B, Caspi R R: Oral tolerance in a murine model of relapsing experimental autoimmune uveoretinitis (EAU): induction of protective tolerance in primed animals. Clinical and experimental immunology 1997, 109 (2):370-376.
64. Chan C C, Caspi R R, Ni M, Leake W C, Wiggert B, Chader G J, Nussenblatt R B: Pathology of experimental autoimmune uveoretinitis in mice. Journal of autoimmunity 1990, 3 (3):247-255.
65. Kezuka T, Takeuchi M, Keino H, Usui Y, Takeuchi A, Yamakawa N, Usui M: Peritoneal exudate cells treated with calcitonin gene-related peptide suppress murine experimental autoimmune uveoretinitis via IL-10. Journal of immunology 2004, 173 (2):1454-1462.
66. Joussen A M, Murata T, Tsujikawa A, Kirchhof B, Bursell S E, Adamis A P: Leukocyte-mediated endothelial cell injury and death in the diabetic retina. Am J Pathol 2001, 158 (1):147-152.
67. Okunuki Y, Usui Y, Nagai N, Kezuka T, Ishida S, Takeuchi M, Goto H: Suppression of experimental autoimmune uveitis by angiotensin II type 1 receptor blocker telmisartan. Invest Ophthalmol Vis Sci 2009, 50 (5):2255-2261.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating autoimmune uveitis in a subject, the method comprising administering to a subject in need thereof a therapeutically effective amount of a CSF1R inhibitor.

2. The method of claim 1, wherein the inhibitor of CSF1R is selected from the group consisting of PLX647; Ki20227; Pexidartinib; PLX7486; OSI-930; Linifanib; ARRY-382; JNJ-40346527; GW2580; GTP 14564; AAL-993; BLZ945; Emactuzumab; AMG820; IMC-CS4; and cabiralizumab.

3. The method of claim 1, wherein the inhibitor is administered locally to the eye.

4. The method of claim 3, wherein the inhibitor is administered topically or periocularly.

5. The method of claim 1, wherein the inhibitor is administered systemically.

6. The method of claim 1, further comprising administering a supplementary active compound selected from the group consisting of corticosteroids; antimetabolites; alkylating/cytotoxic agents; T cell and calcineurin inhibitors; IVIG; and immunosuppressant biologicals.

7. A CSF1R inhibitor for use in treating autoimmune uveitis in a subject.

8. The CSF1R inhibitor for the use of claim 7, which is selected from the group consisting of PLX647; Ki20227; Pexidartinib; PLX7486; OSI-930; Linifanib; ARRY-382; JNJ-40346527; GW2580; GTP 14564; AAL-993; BLZ945; Emactuzumab; AMG820; IMC-CS4; and cabiralizumab.

9. The CSF1R inhibitor for the use of claim 7, which is formulated for topical or periocular administration, or for systemic administration.

10. The CSF1R inhibitor for the use of claim 7, which is formulated for administration with a supplementary active compound selected from the group consisting of corticosteroids; antimetabolites; alkylating/cytotoxic agents; T cell and calcineurin inhibitors; IVIG; and immunosuppressant biologicals.
